(19) United States
(12) Patent Application Publication
(10) Pub. No.: US 2026/0279532 A1
(43) Pub. Date: Sep. 17, 2026

(54) SYSTEMS AND METHODS FOR GENERATION OF HEALTH RECOMMENDATIONS

(71) Applicants: Ravi Michael Mohan, Toronto (CA); Matthew Nichol, Aurora (CA)

(72) Inventors: Ravi Michael Mohan, Toronto (CA); Matthew Nichol, Aurora (CA)

(21) Appl. No.: 19/566,227

(22) Filed: Mar. 13, 2026

Related U.S. Application Data
(60) Provisional application No. 63/771,203, filed on Mar. 13, 2025.

(51) Int. Cl. G16H 20/00; A61B 5/00; G16H 10/60

(52) U.S. Cl. CPC G16H 20/00; A61B 5/7282; G16H 10/60

(57) ABSTRACT

Disclosed are systems and methods that generate real-time health recommendations by computing higher-order derivatives, including jerk (third derivative), of physiological parameters and composite indices to detect early trend inflections that may indicate changes in health conditions. The systems may include a server with a health recommendation unit, databases, and data models that time-align heterogeneous inputs and optionally smooth signals prior to differentiation. Derivatives may be estimated from irregularly sampled clinical data. Events may be detected using magnitude thresholds, sign changes, and persistence windows, triggering intervention, confirmation, or monitoring-cadence recommendations. Examples include LDL cholesterol trajectories and allostatic load (AL) methods that select, normalize, and weight multi-system physiological parameters to determine an allostatic load index AL(t), followed by computation of one or more derivatives of AL(t) including at least a third derivative. Outputs may used to feed a closed-loop that adapts thresholds, weights, and goal difficulty to personalize future guidance. The approach enables earlier, explainable recommendations than absolute levels alone.

### CROSS REFERENCE TO RELATED APPLICATIONS

This application claims the benefit of U.S. Provisional Application 63/771,203 filed on Mar. 13, 2025, which is incorporated by reference herein in its entirety.

### FIELD

The present disclosure generally relates to health recommendation systems and methods, and in particular, generating health recommendations based on higher order derivatives for physiological parameters.

### INTRODUCTION

The following is not an admission that anything discussed below is part of the prior art or part of the common general knowledge of a person skilled in the art.

Healthcare systems struggle to help individuals prevent health challenges and incidents. Frequently, the first indication of a serious health problem is a serious symptom that is not detected until the condition has become acute. One cause of this is the limited information revealed by reviewing values for various physiological parameter either in isolation, or in combination as a snapshot at a single timepoints or a simple comparison between physiological parameter values at two or a few timepoints.

There is a need for systems and methods that analyze physiological parameters, which may also be referred to as physiological values or biomarkers and other terms, to identify health conditions earlier.

### SUMMARY

Healthcare recommendation systems focus on targeted preventative strategies that limit the need for complex healthcare interventions. Generally, these systems are personalized, integrating diverse data inputs to provide a holistic analysis of a particular individual. Data inputs can be objective such as medical history, bloodwork, and body composition; or subjective in nature such as perceived stress evaluation, or perceived sleep quality. Blending the analysis of both subjective and objective inputs can provide a more accurate representation of an individual's well-being, leading to more personalized recommendations for improving an individual's health.

Healthcare prevention systems can also monitor data inputs over a period of time. By analyzing data over time, these systems can determine trends and inform an individual about the effectiveness of their current routines. Upon detecting unfavorable trends, healthcare prevention systems can recommend alternatives to better address an individual's underlying health issues.

In one aspect, the disclosed embodiments provide a computer-implemented method for generating a health recommendation, comprising: receiving, by one or more processors, time-stamped measurements of at least one physiological variable associated with an individual; computing, from the measurements, at least a third derivative (jerk) of the physiological variable with respect to time; detecting a derivative event based at least in part on the jerk, the derivative event comprising one or more of: (i) a magnitude-threshold condition, (ii) a sign change of the jerk, and (iii) satisfaction of a persistence window over a defined duration; and in response to detecting the derivative event, generating a health recommendation selected from an intervention recommendation or a confirmation recommendation, and causing presentation of the health recommendation on a user device.

In some embodiments, the method further includes computing, from the measurements, at least one additional derivative and detecting the derivative event based, at least in part, on both the jerk and the at least one additional derivative.

In some embodiments, the physiological variable comprises one or more of LDL cholesterol, blood pressure, heart-rate variability, blood glucose, resting heart rate, sleep quality, skin temperature, or a composite index thereof.

In some embodiments, the method further includes computing the derivatives comprises applying at least one of: secant-based first-derivative estimation, successive differences for higher derivatives, or analytic derivatives of a smooth fit to the measurements.

In some embodiments, detecting the derivative event comprises determining that an absolute jerk magnitude fall within a magnitude-threshold condition.

In some embodiments, the persistence window requires the jerk condition to be satisfied for a selected number of consecutive estimates or for at least a duration of time.

In some embodiments, the method further includes time-aligning data from heterogeneous sources and optionally applying smoothing using a moving average or spline prior to computing the derivatives.

In some embodiments, the health recommendation comprises continuing with current therapy when the jerk satisfies a decreasing or near-zero condition for the persistence window.

In some embodiments, the method further includes visualizing the physiological variable and at least one derivative with threshold overlays and pairing the visualization with the recommendation.

In some embodiments, the method further includes updating a monitoring cadence based on the derivative event.

In some embodiments, the method further includes computing derivatives includes interpolating a derivative series to a regular evaluation grid to support daily recommendations.

In some embodiments, the measurements are irregularly sampled clinical values and the derivatives are computed using actual elapsed time between samples.

In some embodiments, the method further includes the derivative event is detected on a composite index computed from multiple physiological variables.

In some embodiments, the recommendation includes intervention adjustment when the jerk indicates erratic or concerning dynamics.

In some embodiments, the method is performed by a server comprising a health recommendation unit configured to communicate with user devices via a network.

In another aspect, there is provided a system comprising one or more processors and memory storing instructions that, when executed, cause the system to perform the method described herein, and optionally, a health recommendation unit configured to deliver the recommendation to a user device.

In another aspect, there is provided a computer-implemented method for estimating derivatives from irregularly sampled physiological data, comprising: receiving time-stamped measurements (tᵢ, xᵢ) of a physiological variable with non-uniform intervals; computing first-derivative estimates as secant slopes on intervals [tᵢ₋₁, tᵢ] and associating each first-derivative value with an interval midpoint; computing second- and third-derivative estimates by successive differences between derivative values divided by actual elapsed time between corresponding derivative timestamps; and interpolating at least one derivative series to arbitrary query times to support generation of time-normalized recommendations.

In some embodiments, the method further includes smoothing prior to differentiation using at least one of a moving average or a spline fit.

In some embodiments, interpolating comprises linear interpolation, a monotone interpolant, or a Hermite interpolant that preserves qualitative trends.

In some embodiments, the method further includes analytically from a smooth function S(t) fitted to (tᵢ, xᵢ), including S′(t), S″(t), and S″(t).

In some embodiments, the derivatives are provided to a derivative-event detector configured to apply threshold, sign-change, and persistence logic.

In another aspect, there is provided a computer-implemented method for stress-related health recommendations, comprising: selecting a plurality of physiological parameters across at least two of metabolic, cardiovascular, and inflammatory systems; normalizing and optionally weighting the parameters to form a time-stamped allostatic load index AL(t); computing at least a third derivative (jerk) of the allostatic load index AL(t); detecting a derivative event on at least on the third derivative; and generating a stress-related health recommendation based on the derivative event.

In some embodiments, the method further includes computing at least one additional derivative of the allostatic load index AL(t) and detecting the derivative event based, at least in part, on both the jerk and the at least one additional derivative.

In some embodiments, the parameters include at least two of: C-reactive protein (CRP), glycated hemoglobin (HbA1c), total cholesterol, high-density lipoprotein (HDL), systolic blood pressure, diastolic blood pressure, resting heart rate, serum albumin, and a central adiposity measure.

In some embodiments, the central adiposity measure is based on a measurement of visceral fat.

In some embodiments, the allostatic load index AL(t) is a continuous score obtained by summing direction-aligned, normalized parameter values, optionally weighted by relevance.

In some embodiments, the method further includes validating AL(t) against a subjective stress scale and/or clinical outcomes.

In some embodiments, a derivative event comprises a jerk magnitude threshold satisfied for a persistence window.

In some embodiments, the method further includes reinforcing a current routine when jerk indicates sustained stabilization, or intensifying interventions when jerk indicates concerning dynamics, and/or modifying monitoring cadence.

In some embodiments, the method further includes harmonizing the time bases of the physiological parameters by interpolating parameter values to a series of periodic timepoints before computing AL(t).

In some embodiments, the derivative computation uses actual elapsed time between observations and optionally uses spline-based analytic derivatives of AL(t).

In some embodiments, the recommendation is displayed together with a visualization of AL(t) and at least one derivative with threshold bands.

In another aspect, there is provided a computer-implemented method for closed-loop health personalization, comprising: generating a health recommendation based on a derivative event detected from a physiological variable or index; ingesting feedback including subsequent measurements and user or clinician responses; and updating at least one model parameter selected from: a threshold, a persistence duration, a parameter weight, or a goal difficulty level, such that future recommendations are personalized based on the feedback.

In some embodiments, the method further includes adjusting goals automatically when an adherence or lifestyle score improves beyond a threshold for a defined interval.

In some embodiments, the method further includes updating model parameters are specific to particular physiological parameters.

In some embodiments, the feedback ingestion incorporates a composite index as an input to subsequent derivative analysis.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1 shows a schematic diagram of an example health recommendation system in accordance with some embodiments.

FIG. 2 illustrates a server for an example health recommendation system in accordance with some embodiments.

FIG. 3 shows an illustration of an example data flow for an example health recommendation system in accordance with some embodiments.

FIG. 4 shows an illustration of an example analysis executed on a physiological parameter for a health recommendation system in accordance with some embodiments.

FIG. 5 illustrates a smoothed plot of measurement of LDL cholesterol at regular sampling intervals.

FIGS. 6A-C illustrate estimation of derivatives from irregularly sampled physiological data and for interpolating derivative estimates at arbitrary times.

FIGS. 7A-D illustrate an analysis of therapy using higher order derivatives including a third derivative.

FIG. 8 illustrates a method for derivative event identification.

FIG. 9 illustrates a method for assembling a physiological index.

### DESCRIPTION OF VARIOUS EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

It should also be noted that the terms “coupled” or “coupling” as used herein can have several different meanings depending on the context in which these terms are used. For example, the terms coupled or coupling can have a logical, mechanical, fluidic or electrical connotation. For example, as used herein, the terms coupled or coupling can indicate that two elements or devices can be directly connected to one another or connected to one another through one or more intermediate elements or devices via an electrical or magnetic signal, electrical connection, an electrical element or a mechanical element depending on the particular context. Furthermore, coupled electrical elements may send and/or receive data.

Unless the context requires otherwise, throughout the specification and claims which follow, the word “comprise” and variations thereof, such as, “comprises” and “comprising” are to be construed in an open, inclusive sense, that is, as “including, but not limited to”.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Exemplary communicative pathways include, but are not limited to, electrically conductive pathways (e.g., electrically conductive wires, electrically conductive traces), magnetic pathways (e.g., magnetic media), optical pathways (e.g., optical fiber), electromagnetically radiative pathways (e.g., radio waves), or any combination thereof. Exemplary communicative couplings include, but are not limited to, logical couplings, electrical couplings, magnetic couplings, optical couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

The example systems and methods described herein may be implemented as a combination of hardware or software. In some cases, the examples described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a keyboard, mouse, touchscreen, or the like), and at least one output device (e.g. a display screen, a printer, a wireless radio, or the like) depending on the nature of the device.

Some elements that are used to implement at least part of the systems, methods, and devices described herein may be implemented via software that is written in a high-level procedural language such as object-oriented programming. The program code may be written in C⁺⁺, C#, JavaScript, Python, or any other suitable programming language and may comprise modules or classes, as is known to those skilled in object-oriented programming. Alternatively, or in addition thereto, some of these elements implemented via software may be written in assembly language, machine language, or firmware as needed. In either case, the language may be a compiled or interpreted language.

At least some of these software programs may be stored on a computer readable medium such as, but not limited to, a ROM, a magnetic disk, an optical disc, a USB key, and the like that is readable by a device having at least one processor, an operating system, and the associated hardware and software that is used to implement the functionality of at least one of the methods described herein. The software program code, when read by the device, configures the device to operate in a new, specific, and predefined manner (e.g., as a specific-purpose computer) in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

Various physiological parameters can be monitored to provide a wholistic picture of an individual's health. For example, parameters such as height, weight, age can provide a high-level overview of an individual's health at a discrete instance in time. These values can also be compared with threshold values (e.g., Body Mass Index (BMI)) to provide an indication of an individual's health compared to the average population within their age and gender.

While it may be relatively straightforward to calculate an individual's BMI—and determine the relevant constituent inputs (e.g., height and weight)—it can be increasingly challenging to pinpoint which parameters contribute to other physiological measurements that cannot be easily quantified such as stress. This challenge can be magnified when subjective factors such as perceived experience, which varies between individuals, are considered. Further, there may be underlying relationships between data inputs which may contribute to increased stress, but not well researched in the literature. Accordingly, there is a need for a system which considers diverse data inputs and determines its relative contribution to a particular health condition.

Various parameters can also be measured over a period of time to provide a high-level overview of an individual's health over that time period. For example, an individual's weight can be tracked over time to determine whether they are gaining weight or losing. Based on the information, an individual can plan how improve the quality of their life based on their specific goals.

Measuring the change in a parameter over time (i.e., a first derivative such as velocity) can provide a trend for that specific parameter. However, it can also be useful to measure the change in velocity over time for a single parameter (i.e., a second derivative such as acceleration) or the change in acceleration over time for that parameter (i.e., jerk). The acceleration of a parameter can provide the rate of change for a particular trend, and the jerk of a parameter can provide the rate of change of the acceleration. For example, a steady acceleration in weight loss can indicate the compounding effects of lifestyle changes over a given period of time. In the same way, higher order derivatives—such as velocity, jerk, and fourth and higher derivatives—may provide further insights into an individual's health profile, such as early indicators that may signal the effectiveness of an intervention. Accordingly, there is a need for a system which considers higher order derivatives for physiological parameters in order to provide for an individual more accurate and beneficial health recommendations.

Although systems can be trained to calculate higher-order derivatives of physiological parameters, it can be difficult to glean meaningful insights from these values as they relate to an individual's health and lifestyle-even for trained physicians. For example, it can be straightforward to identify when a physiological parameter is outside of a normal range or whether it is trending unfavorably. However, it can be challenging to pinpoint which aspects of a treatment plan are effective for shifting the parameter within the normal range-particularly, on an individual basis.

In addition, it can be difficult for an individual to know whether they should take actionable steps to improving their health without guidance from a physician. For example, if an individual's physiological parameters are within the normal range- and do not need immediate intervention-a physician may not guide an individual to change their lifestyle. A physician may only contact an individual if the physician notices that the individual's health is worsening. However, earlier interventions could have potentially been adopted to prevent a decline in health entirely if the individual had been informed sooner. Accordingly, there is a need for a system which provides early indicators for changes in an individual's health as well as immediate feedback with respect to how an individual can improve their health.

The described embodiments that follow relate to systems and methods for generating health recommendations. The health recommendations can be based on an analysis of higher order derivates for a physiological variable. By way of example, the systems and methods disclosed herein can be used in industries such as healthcare. For example, an individual may desire health recommendations to improve their quality of life. A physician may reference health recommendations when creating a treatment/preventative plan for a patient.

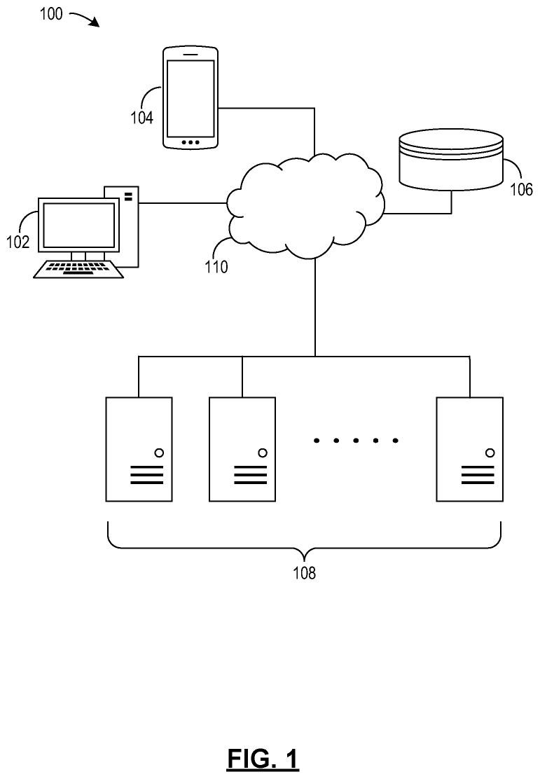

Reference is first made to FIG. 1, which shows a schematic diagram of an example health recommendation system. The system 100 shows a health recommendation system that can be used to provide health recommendations to an individual based on various inputs. The system 100 may include a server 102, one or more user devices 104, a database 106, and one or more data models 108. The server 102, the one or more user devices 104, the database 106, and the one or more data models 108 can be connected to each other through network 110.

The one or more user devices 104 can be configured to receive inputs from a user. The inputs can be various physiological variables, such as but not limited to medical history, current lifestyle, exercise test results, body composition data, and bloodwork. The one or more user devices can be, for example, a smart device, a smart wearable, a health tracker, or any device configured to measure physiological data from a user.

The database 106 can be a memory for storing received inputs. Received inputs can be from a user or an external database (e.g., from a public health record system) that stores health information about a user.

The one or more data models 108 can be configured to analyze the received inputs and generate outputs (i.e., health recommendations). For example, the one or more data models 108 can be configured to perform a higher order derivative analysis (i.e., higher than a second order derivative) on one or more of the received inputs. The one or more data models 108 can be configured to analyze a single variable or multiple variables in combination.

The network 110 may be any network capable of carrying data, which may be connected via infrastructure such as Ethernet, RS-485, PROFIBUS, CAN Bus, USB, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, and others, including any combination of these, capable of interfacing with, and enabling communication between any or all of the server 102, the one or more user devices 104, the database 106, and/or the one or more data models Each of the server 102, the one or more user devices 104, the database 106, and/or the one or more data models 110, may be equipped with suitable network communication hardware so as to enable communications with each other through protocols such as MODBUS, IEEE 802.3, IEEE 802.11, DeviceNet, or any other communications protocol suitable for communication between industrial devices.

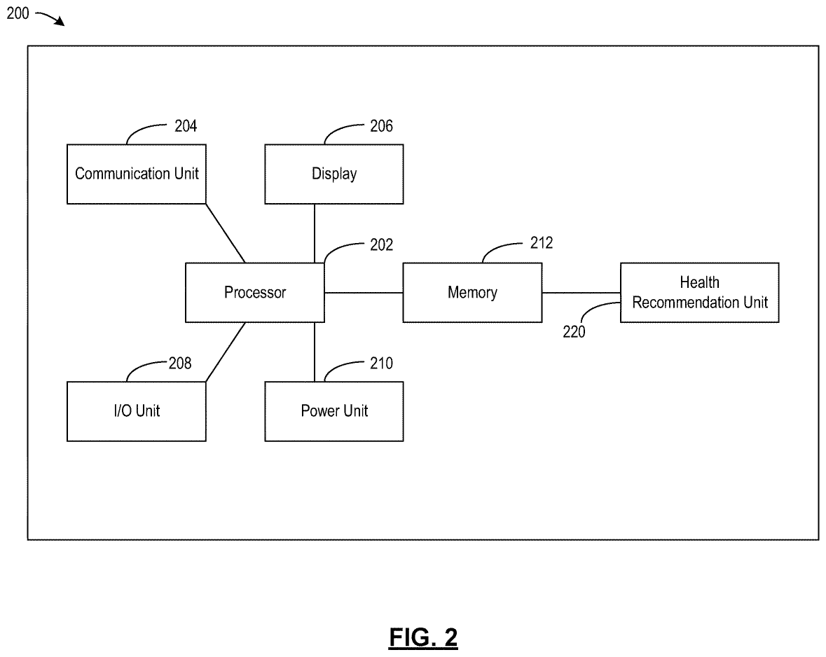

Reference is next made to FIG. 2, which shows a device diagram of a server for an example health recommendation system in accordance with one or more embodiments. The server 102 can include a processor unit 202, a communication unit 204, an I/O unit 208, a power unit 210, and a memory 212.

The communication unit 204 can include any combination of hardware that enables wired or wireless connection capabilities. For example, the communication unit 204 can include a radio or network card that communicates using standards such as IEEE 802.3, IEEE 802.11, DeviceNet, MODBUS, and any other protocol suitable for communicating with industrial devices. The communication unit 204 can allow the server 102 to communicate with the one or more devices 104, user database 106, the database 108, the one or more data models 110, and other devices, computers, local area networks, wide area networks, and external networks.

In the embodiment shown in FIG. 1, server 102 includes hardware enabling communication through the network 110. Server 102 can then communicate to send and receive data, such as received inputs from a smart wearable device to the system, image data, alerts, text data, and more. For instance, server 102 may receive medical history from a user and relay the medical history to the database 108, or the one or more data models 110.

The processor unit 202 controls the operation of the server 102. The processor unit 202 can be any suitable processor, controller or digital signal processor that can provide sufficient processing power depending on the configuration, purposes and requirements of the server 102 as is known by those skilled in the art. For example, the processor unit 202 may be a high-performance general processor, such as an Intel® processor or an AMD® processor. The processor unit 202 may preferably contain a graphics card or a dedicated graphics processing unit (GPU), such as an Nvidia® graphics processor, for accelerating locally run machine learning models. The provision of a GPU may provide speed advantages for running machine learning algorithms such as material verification, object recognition, defect detection, bit verification, and more. In some embodiments, the processor unit 202 can include more than one processor with each processor being configured to perform different dedicated tasks.

The I/O unit 208 can include hardware for interfacing with at least one of a mouse, a keyboard, a touch screen, a thumbwheel, a trackpad, a trackball, a card-reader, an audio source, a microphone, voice recognition software and the like again depending on the particular implementation of the server 102. The I/O unit 208 may then facilitate taking user input to the server 102. In some cases, some of these components can be integrated with one another.

The power unit 210 can be any suitable power source that provides power to the server 102 such as a power adaptor or a rechargeable battery pack depending on the implementation of the server 102 as is known by those skilled in the art.

The memory 212 comprises software code for implementing, among other programs, the health recommendation unit 220. The memory 212 can include RAM, ROM, one or more hard drives, one or more flash drives or some other suitable data storage elements such as disk drives, etc.

The health recommendation unit 220 can generate health recommendations based on an analysis of various inputs from one or more user devices. The analysis can be executed by the one or more data models 110. The health recommendation unit 220 can provide for example, a summary of medical history, primary care recommendations, and preventative care recommendations.

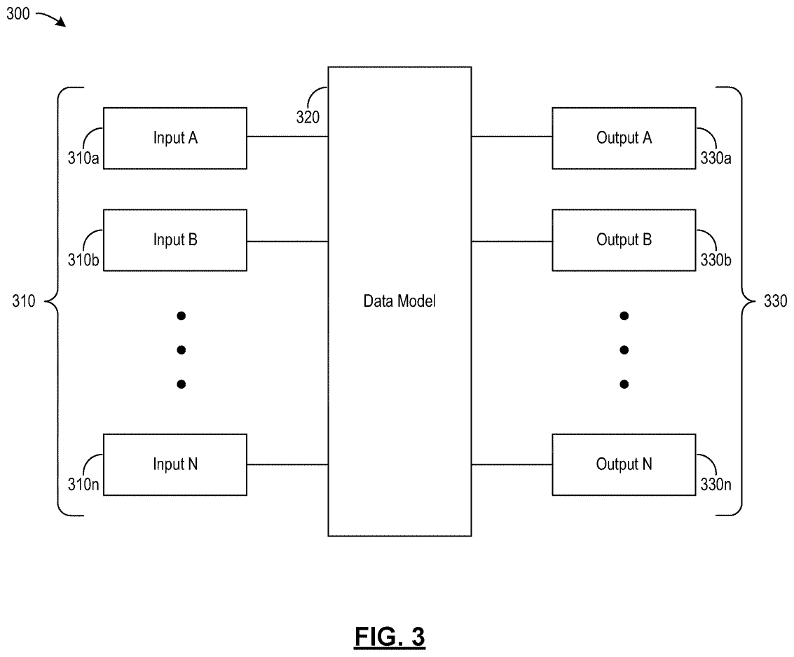

Reference is next made to FIG. 3, which shows an illustration of a data flow for an example health recommendation system in accordance with some embodiments. The system 300 can receive inputs 310. The inputs 310 can be physiological parameters related to an individual. The system 300 can receive inputs 310 for any physiological parameter up to input 310n, where input 310n is the final input provided.

The system 300 can receive inputs which can be objectively quantified or measured (e.g., height, weight, age average daily, weekly or monthly steps, VO₂ max, push up max, plank test, sleep data). The system can also receive inputs that cannot be quantified or measured, but which can be subjectively assessed such as, for example, lifestyle and behavior indicators, stress, or emotional well-being.

The inputs can be immutable (e.g., age, family medical history, genetic disorders) or may change over time (i.e. time-varying) (e.g., body composition data, exercise routine, diet, bloodwork data). Inputs that can change over time can be measured over different timescales depending on the nature of the physiological parameter. For example, it may be practical to measure LDL cholesterol on a monthly basis and blood glucose levels on a daily, hourly or continuous basis. The system 300 can execute a higher-order derivative analysis (e.g., acceleration or jerk analysis) on a time-varying parameter.

As an example, input A 310a can be an individual's medical history which can include, for example, the individual's age, gender, and ethnicity. The individual's medical history can also include current medical conditions, past medical conditions, surgical history, psychological history, current/former medications and supplements, family medical history (e.g., chronic disease, cancer history, genetic disorders), psychosocial index, and cancer screening (e.g., prostate, breast, colon, lung).

As another example, input B 310b can be parameters related to an individual's current lifestyle and behaviors which can include, for example, the individual's social history (e.g., exposure to drugs and alcohol), allergies and sensitivities, diet (e.g., intermittent fasting, Mediterranean, ketogenic, paleo, vegetarian, vegan, low carb), and exercise regimen (e.g., cardiovascular strength, flexibility, balance).

The system 300 can execute an analysis of the inputs 310 using a data model 320. The data model 320 can be configured with one or more machine learning models. The data model 320 can also be configured to consider expert opinion (e.g., physician recommendations, psychologist assessments) and real-time feedback from the individual.

For example, the data model 320 can be configured to create a real time index for allostatic load to provide continuous monitoring for the impact of chronic stress on an individual's health. The data model 320 can receive input data from a health tracking device (e.g., a smart watch, or a smart ring). The system 300 can select parameters based on overlapping metrics between allostatic load indicators (e.g., cortisol levels, heart rate variability, blood pressure, resting heart rate) and input data from the health tracking device (i.e., configured to track, for example, heart rate, heart rate variability, skin temperature, activity levels and motion) to generate a comprehensive measure of allostatic load.

The data model 320 can combine these inputs with any other inputs received by the system (e.g., LDL Cholesterol, waist to hip ratio). The data model 320 can generate a scoring system based on percentile thresholds or clinical guidelines. Each metric can be normalized to a common scale (e.g., 0 to 1). Each metric can also be assigned a weight based on its relevance to allostatic load. For example, heart rate variability may carry more weight due to its link with stress regulation, and sleep quality may be weighted higher for recovery). The normalized and weighted scores can be summed to create an allostatic load index where higher scores may indicate greater physiological dysregulation. The allostatic load index can be validated against subjective evaluations of stress (i.e., received as a data input) such as perceived stress scale or other related health outcomes (e.g., blood pressure, mental health assessments).

The system 300 can generate outputs 330 based on the analysis executed by the data model 320. The outputs 330 can be a health overview as well as health recommendations for an individual. The system 300 can generate outputs 330 for an individual. The outputs 330 can be in various forms depending on the nature of the information presented.

For example, output A 330a can be a medical history summary presented as a summary document or an infographic. The medical history summary can include information related to an individual's current medical conditions, medication summary, or a body composition profile, lifestyle summary. In addition, or alternatively, the medical history summary can be presented in the form of a table representing an individual's lifestyle in categories (e.g., diet, rest, exercise, social/mental, supplement) with an associated scoring system. The scoring system can be automatically populated with the outputs 330 of the system 300. The scoring system can continuously or periodically update as more inputs 310 are received by the system 300 (e.g., through a health tracking device). The scoring system can be used as a benchmark for an individual's progress for achieving a healthier lifestyle. For example, when the individual consistently leads a healthier lifestyle (i.e., by consistently achieving a high score on the scoring system), the scoring system can automatically adapt to provide more challenging goals to ensure the individual is improving on an ongoing basis.

As another example, some outputs 330 can be various preventative recommendations. Preventative recommendations can include for example, a cancer screening strategy which provides an individual's cancer history (i.e., organized by type of cancer screened) as well as recommendations for future screenings. Preventative recommendations can also include prevention strategies related to exercise (e.g., recommended strength, endurance, and stability routines), prevention strategies related to nutrition (e.g., recommended diet and supplement intake), and prevention strategies related to rest (e.g., recommended hours for sleep, mindfulness, and reflection).

Generated outputs can be used as feedback into the system 300 as inputs for further analysis. For example, as described above, the output of an allostatic load index could be provided as an input 310 to system 320. In some embodiments, an allostatic load index can be generated as an output based on inputs such as blood work, clinical history, and health tracker information. The generated allostatic load index can then be used as an input into the system 300 for further analysis. For example, a higher-order derivative analysis can be executed on an individual's allostatic load index over a period of time.

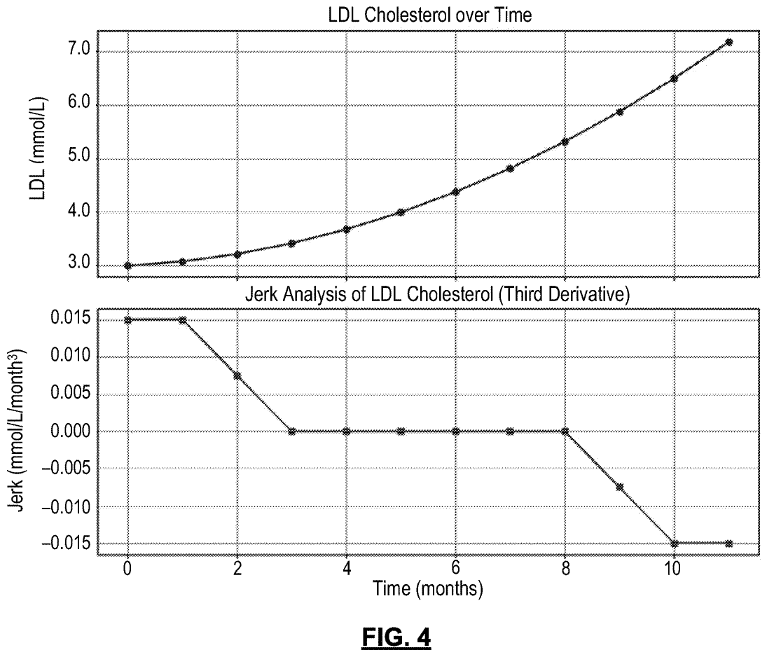

Reference is next made to FIG. 4, which shows an illustration of an example analysis executed on a physiological parameter for a health recommendation system in accordance with some embodiments. The analysis shown in FIG. 4 can be executed by the system using a data model as described herein.

Monitoring the progression of physiological parameters over time can illustrate its potential health impacts. In addition to observing absolute levels (i.e., the value of a physiological parameter at a discrete point in time), analyzing its derivatives—rate of change (first derivative), acceleration (second derivative), and jerk (third derivative)—can also provide valuable insights that can lead to accurate health recommendations. For example, abrupt changes in trajectory of levels for a particular physiological parameter such as low-density lipoproteins (LDL) can signal underlying physiological shifts.

The temporal progression of LDL cholesterol levels, measured in mmol/L (or mg/dl, 1 mmol/L=38.67 mg/dL) and their associated third order derivative analysis (also referred to as a jerk analysis) can be seen in FIG. 4.

The graph titled “LDL Cholesterol over Time” shows a consistent upward trend with an accelerating trajectory. This may suggest that not only are LDL levels increasing, but the rate at which they increase is also growing over time. This may trigger the system to generate a health recommendation to prompt an individual to alter their behavior. For example, the system may provide a notification to an individual displaying the graph in FIG. 4 (i.e., “LDL Cholesterol over Time”) alongside a list of recommended LDL-rich foods to avoid.

The graph titled “Jerk Analysis of LDL Cholesterol (Third Derivative)” shows an initial positive value followed by stabilization at zero (i.e. between months three to eight), indicating a consistent acceleration during this period. During months eight to ten, a negative jerk is observed, signaling a decrease in the rate of acceleration. The stabilization of jerk values during months three to eight indicates that while LDL cholesterol levels are increasing, sudden shifts or disruptions are minimal for most of the observed period. The reduction in jerk to zero during months three to eight, and the negative jerk observed during months eight to ten could indicate physiological or external interventions affecting cholesterol metabolism. While the absolute cholesterol level continues to increase, and the velocity of the cholesterol levels is still positive, the decreasing jerk provides a relatively early indication that such physiological or external interventions are having an effect on the trend of the person's cholesterol levels. This may trigger the system to generate a health recommendation to prompt an individual to understand that their interventions are having a beneficial effect, and that they should continue the interventions. Such a recommendation may be generated much earlier than would be notable from the absolute cholesterol level. For example, the system may provide a notification to an individual displaying the graph in FIG. 4 (i.e., “Jerk Analysis of LDL Cholesterol (Third Derivative)”) alongside a recommended exercise plans that could help minimize LDL cholesterol levels further. The system may be able to provide this notification as early as month 2 when the jerk declined. In some embodiments, the system may be configured to provide such a notification only when the change in jerk exceeds a threshold, when the jerk has become negative, when the jerk has been consistently reduced from its initial level for a selected time period, when the jerk has been consistently decreasing for a selected time period, or a combination of these and other criteria selected to identify an improvement in the individual's cholesterol trends, even though their absolute cholesterol may still be increasing.

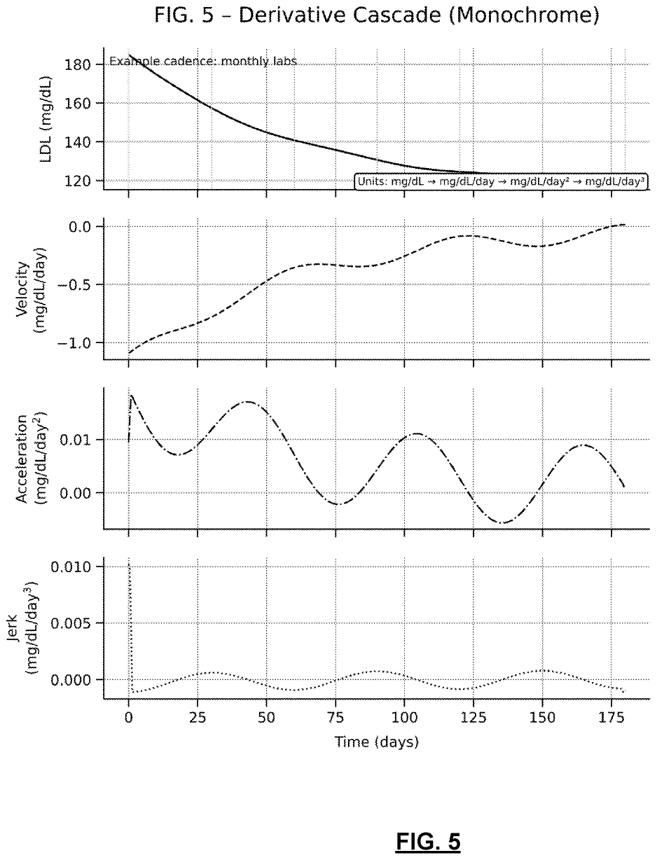

FIG. 5 illustrates a smoothed plot of measurement of LDL cholesterol at regular sampling intervals. The sampling cadence for different physiological variables may vary depending on the variable and the type and availability of testing equipment. The results may be plotted and mathematical techniques such as regression may be applied to plot a smooth line based on the measured values for the physiological variable. Typical clinical sampling cadences for different physiological variables may be annual (or longer than a year) semi-annually, monthly, daily or continuous sampling using a wearable sampling device such as a smartwatch. The smoothed line may account for sparse data or low frequency intervals. Visual callouts highlight how subtle structure that may be hard to detect in x(t) can appear more prominently as features in v(t), a(t), and especially in j(t), which tracks rapid changes in acceleration and is therefore highly sensitive to early shifts in trend. This sensitivity enables earlier recommendations (e.g., “continue current therapy” when jerk turns and persists in a favorable direction) than approaches that rely solely on absolute levels. In FIG. 5, the acceleration a(t) shows greater variability than the base LDL level (x(t), while the jerk j(t) shows that the changes in acceleration are not particularly marked, but are rather small. The combination of the acceleration and jerk plots indicates that the person's LDL cholesterol is changing relatively steadily, in this case, declining in a favourable trajectory. This indicates that the person's efforts to reduce their LDL cholesterol appear to be successful and may allow a clinician to advise the person to continue with their efforts.

In some embodiments, the derivatives of a composite index (e.g., allostatic load) in addition to single parameters; the same derivative semantics apply and support derivative-event detection across individual and composite signals.

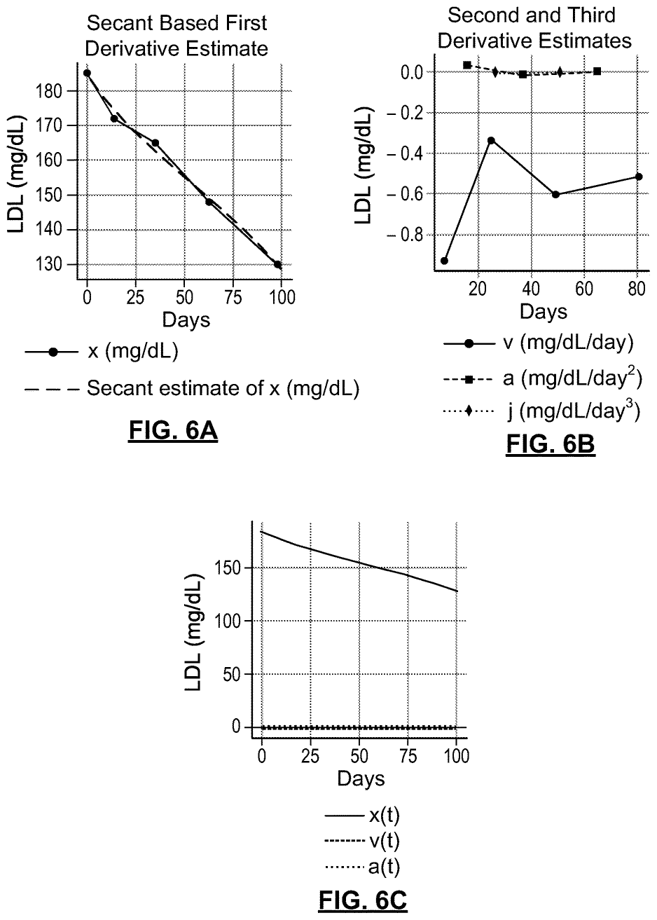

In some situations, tests to determine the value of a physiological value may be performed on an irregular basis. FIG. 6 illustrates several methods for estimating first, second, and third derivatives from irregularly sampled physiological data and for interpolating those derivative estimates at arbitrary times, including times between sampling times (e.g., for daily decision support). The figure comprises three panels: FIG. 6A illustrates secant-based first-derivative estimates on irregular intervals; FIG. 6B constructs second- and third-derivative estimates via successive differences on the native (irregular) grid; and FIG. 6C demonstrates a smooth-function fit (e.g., spline/polynomial) that supports analytic derivatives at any time t. Each panel emphasizes the use of actual elapsed time (not rounded or assumed) between measurements, as clinical sampling is typically non-uniform.

Referring to FIG. 6A, an approach of using secants on irregular intervals is illustrated to estimate first derivatives. The observed samples form a set of sample

$${\{ ( {t}_{i} , {x}_{i} ) \}}_{i = 0}^{N}$$

with non-uniform spacings Δtᵢ=tᵢ−tᵢ₋₁ The first derivative (velocity) is estimated on each interval [tᵢ₋₁, tᵢ] by the secant slope

$${v}_{i} \approx \frac{{x}_{i} - {x}_{i - 1}}{{t}_{i} - {t}_{i - 1}} ( units : \frac{mg / dL}{day} ( for example ) ) .$$

This yields derivative estimates at interval midpoints

$$\text{?} = \frac{1}{2} ( {t}_{i - 1} + {t}_{i} ) ,$$

preserving true time intervals and avoiding bias from assuming a uniform grid. In the LDL example, early intervals (e.g., 0→14 days vs. 14→35 days) produce distinct vᵢ magnitudes reflective of actual scheduling differences.

FIG. 6b illustrates the estimation of Higher-Order Derivatives using Native-Grid Interpolation. From the midpoint-anchored {(\tilde{t}ᵢ, vᵢ)}, the second derivative (acceleration) is estimated between consecutive velocity points using the actual elapsed time between those velocity timestamps:

$${a}_{i} \approx \frac{{v}_{i} - {v}_{i - 1}}{{\tilde{t}}_{i} - {\tilde{t}}_{i - 1}} ( units : \frac{mg / dL}{{day}^{2}} ( for example ) ) .$$

producing acceleration values at acceleration midpoints

$${\hat{t}}_{i} = \frac{1}{2} ( {\tilde{t}}_{i - 1} + {\tilde{t}}_{i} ) .$$

The third derivative (jerk) follows analogously from consecutive acceleration points:

$${j}_{i} \approx \frac{{a}_{i} - {a}_{i - 1}}{{\hat{t}}_{i} - {\hat{t}}_{i - 1}} ( units : \frac{mg / dL}{{day}^{3}} ( for example ) ) .$$

This successive-difference construction propagates the true irregular cadence through all derivative order and avoids timing inaccuracy introduced with each successive differentiation step.

The system may estimate derivative values at times t that do not coincide with the native derivative timestamps (e.g., to generate a daily or other periodic recommendation). This may done in several ways.

Estimated derivatives may be interpolated directly from native-grid. After computing

$$\{ ( {\tilde{t}}_{i} , {v}_{i} ) \} , \{ ( {\hat{t}}_{i} , {a}_{i} ) \} , and \{ ( {t}_{i}^{( j )} , {j}_{i} ) \}$$

on the native grid, the system interpolates each derivative series to any desired t that lies between two available derivative timestamps. The system may estimate a derivative using linear interpolation:

$$\hat{v} ( t ) = {v}_{i} + \frac{{v}_{i + 1} - {v}_{i}}{{\tilde{t}}_{i + 1} - {\tilde{t}}_{i}} ( t - {\tilde{t}}_{i} )$$

$$\hat{a} ( t ) = {a}_{i} + \frac{{a}_{i + 1} - {a}_{i}}{{\tilde{t}}_{i + 1} - {\tilde{t}}_{i}} ( t - {\tilde{t}}_{i} )$$

$$\hat{j} ( t ) = {j}_{i} + \frac{{j}_{i + 1} - {j}_{i}}{{\tilde{t}}_{i + 1} - {\tilde{t}}_{i}} ( t - {\tilde{t}}_{i} )$$

Linear interpolation preserves units and is stable under sparse sampling. In some embodiments, higher-order Hermite or shape-preserving (e.g., monotone) approaches may be used to maintain qualitative trends.

FIG. 6c illustrates another approach of fitting a smooth function to x(t) and evaluate analytic derivatives. The system may fit a smooth curve S(t) to {(tᵢ, xᵢ)}. For example, the system may fit a cubic spline or local polynomial using the actual irregular timestamps as sample locations. Derivatives are then obtained analytically at any t:

$$\hat{v} ( t ) = {S}^{′} ( t ) , \hat{a} ( t ) = {S}^{″} ( t ) , \hat{j} ( t ) = {S}^{′″} ( t )$$

This approach provides smooth, differentiable estimates across gaps and missing data, and is well-suited when noise warrants smoothing before differentiation.

Accurate interpolation on irregular data allows the system to evaluate derivative events, such as threshold crossings, sign changes, and persistence windows, on a consistent evaluation grid (e.g., daily or other time period). This ensures that higher-order signals (such as jerk) can trigger early interventions or “continue-what-works” confirmations at clinically meaningful times, even when measurements arrive sporadically.

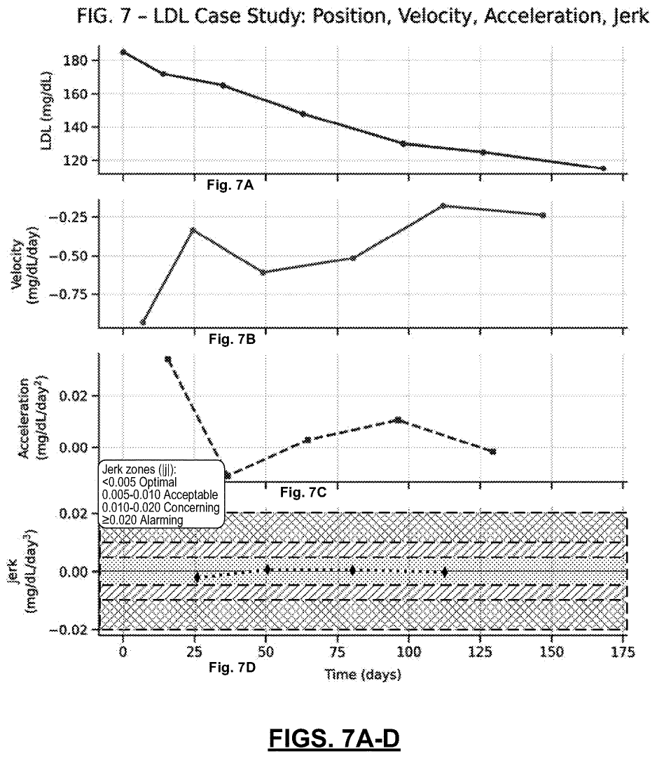

FIG. 7 illustrates an example in which higher-order derivatives, including the third derivative allows earlier and reliable confirmation that therapy is effective, enabling the system to guide patient behavior before absolute LDL levels alone would justify a change in plan.

FIG. 7 comprises four vertically aligned plots that illustrate the evolution of LDL cholesterol under statin therapy and the corresponding first (velocity), second (acceleration), and third (jerk) derivatives computed on irregularly sampled clinical data. The dataset represents seven measurements over twenty-four weeks with the following time/value pairs (days from baseline, LDL in mg/dL):

FIG. 7A the following time and LDL cholesterol value pairs: {(0,185), (14,172), (35, 165), (63, 148), (98, 130), (126, 125), (168,115)}. \{(0,185), (14,172), (35, 16 5), (63,148), (98,130), (126, 125), (168,115)\}{(0,185), (14,172), (35,165), (63, 148), (98, 130), (12 6, 125), (168,115)}.

The times (in days) reflect typical clinic visit variability rather than uniform intervals. Actual elapsed time is used for all derivative calculations shown in FIG. 7 to compensate for the irregular times between clinic visits.

FIG. 7A plots LDL concentration versus time with markers at each clinical sample time and a trend line. The curve exhibits a rapid early decline from 185→172 mg/dl in the first 14 days, followed by a continued decrease to 115 mg/dl by day 168, with visibly diminishing marginal reductions after ~day 98—consistent with a therapeutic response approaching a new steady state. This panel establishes the raw, clinically observable trend that underlies the derivative analysis in FIGS. 7B-D.

FIG. 7B shows first derivative (velocity) v(t) estimates at the midpoints of each measurement interval, computed as the secant slope using actual elapsed time.

Representative values align with three physiologically meaningful phases: (i) a large negative rate in the first interval (≈−0.929 mg/dL/day; days 0-14) indicating rapid drug response; (ii) moderate negative rates through the middle intervals (e.g., ≈−0.607 and −0.514 mg/dL/day) as the patient continues to improve; and (iii) small negative rates late in therapy (≈−0.179 to −0.238 mg/dL/day) reflecting plateau behavior. The use of mid-interval timestamps ensures each velocity point is placed where the underlying slope best represents local behavior between measurements.

FIG. 7C plots second derivative (acceleration) a(t) estimates derived from consecutive velocity estimate points using the actual elapsed time between velocity timestamps (midpoints of adjacent clinical intervals).

These values oscillate with small magnitude around zero (e.g., +0.034, −0.011, +0.003, +0.011, −0.002), indicating fine-scale adjustments in how quickly LDL is declining, rather than dramatic swings. Clinically, such small accelerations are expected as homeostatic systems and pharmacodynamics settle toward equilibrium; this pattern supports a narrative of stable, predictable response rather than erratic behavior.

FIG. 7D plots third derivative (jerk) j(t) estimates derived from consecutive acceleration estimates with the actual elapsed time between acceleration timestamps.

FIG. 7 reveals several features of the patients clinical response to statin therapy in the early and subsequent time periods illustrated:
A distinct negative jerk around the transition from the early rapid-response phase to the subsequent steady decline phase (≈−0.0021 mg/dL/day³), indicating that the acceleration itself is falling—i.e., the big early gains are tapering in a smooth, controlled way, as expected when therapy is effective and the system moves toward a plateau.
Small magnitude jerk values thereafter (e.g., +0.0005, +0.0003, −0.0004 mg/dL/day³), which plot near zero, signaling smooth stabilization rather than oscillatory or chaotic dynamics. This “near-zero jerk” corridor is shown in FIG. 7D in several magnitude threshold bands based on the absolute value of jerk:
         |j|<0.005 (Optimal);
   0.005≤|j|<0.010 (Acceptable);
   0.010≤|j|<0.020 (Concerning); and
         |j|≥0.020 (Alarming).

FIG. 7D illustrates that when jerk decreases and remains small in magnitude over a defined persistence interval (e.g., N consecutive derivative estimates or T days), the system can confidently present a “continue current therapy” recommendation-even if absolute LDL has not yet reached goal. In this manner jerk provides an early indicator that the intervention is working as intended. This persistence rule maps directly to the system's derivative event logic and recommendation process.

FIG. 7 illustrates the benefit of determining derivatives based on actual elapsed time. Because clinic visits in this case occur at non-uniform intervals (e.g., 14, 21, 28, 35, 28, and 42 days between successive draws), all derivatives are computed using actual elapsed time at each step (interval midpoints for velocity, then midpoints-of-midpoints for acceleration, etc.). This treatment ensures that the magnitudes and timing of v, a and j accurately reflect the patient's trajectory and are not biased by an assumed regular timing interval between clinic visits and blood draws.

The jerk-based early indicator behavior illustrated in FIG. 7D operationalizes the broader system architecture: ingestion of multi-modal inputs, derivative computation on a precise time base, event detection via thresholds/sign changes/persistence, and automated recommendations (intervention vs. confirmation) with closed-loop feedback to update future analyses.

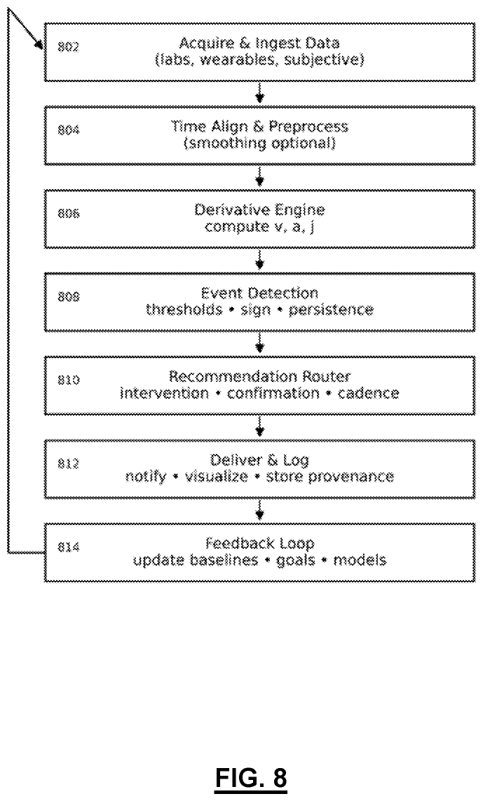

FIG. 8 illustrates a method for streamlined derivative event decision that converts multi-modal health inputs into real time recommendations using higher-order derivatives, and then feeds outcomes back to improve subsequent analyses. The pipeline may be implemented by the health recommendation unit running on server 102 (FIG. 2) and coupled to data models 108 (FIG. 1).

At 802, incoming signals include objective measurements (e.g., bloodwork such as LDL, BP, glucose; wearable features such as HR, HRV, skin temperature, activity) and subjective inputs (e.g., perceived stress, sleep quality checks) are ingested into the system. Data may arrive at irregular cadences (clinic visits vs. continuous wearables). The system records exact timestamps and source metadata to enable time-accurate derivative estimation downstream.

At 804, incoming data signals are time-aligned across sources; missing data may be imputed, interpolated or flagged. Noise mitigation may optionally be applied prior to differentiation (for example, moving averages or spline smoothing techniques may be used to smooth noise in signals).

At 806, for each time-varying variable or composite index (e.g., allostatic load), the system computes at least one or more of v(t), a(t), j(t) and optionally higher order derivatives.

With physiological variable that are irregularly sampled, the engine may use secants for first derivatives and successive differences for higher derivatives—anchoring each estimate to the true elapsed time between timestamps. A smooth fit (e.g., cubic spline) may be used to estimate derivatives at any arbitrary time.

At 808, the system evaluates derivative events including: Magnitude thresholds (|j|<0.005, etc.), Sign changes (e.g., jerk turning positive or negative indicating an acceleration or deceleration of the change of a physiological variable or composite index), Persistence windows (event must hold for n consecutive estimates or T days), etc.

Thresholds may be parameter-specific and time-unit specific (per day vs. per week) and can adapt over time for a user cohort, with clinician override.

At 810, detected events are mapped to recommendation categories, for example:
Intervention/Adjustment: escalate therapy, review interactions, modify plan when jerk indicates erratic or concerning dynamics.
Confirmation/Reinforcement: “Continue what's working” when jerk indicates sustained stabilization despite absolute values not yet at target.
Monitoring Cadence: adjust sampling frequency (e.g., increase monitoring in concerning jerk zones).

At 812, recommendations and visuals (e.g., LDL with jerk overlay and thresholds) are delivered to users/clinicians. The system logs data snapshots, derivative states, event criteria, and the issued recommendation for auditing, learning, and regulatory traceability.

At 814, outputs (e.g., updated allostatic load, adherence indicators, response classification) are input to the system new inputs. The system may adjust goals as scores improve (or change), re-learn thresholds, and refine model weights based on user/clinician feedback. This closed-loop design supports ongoing personalization.

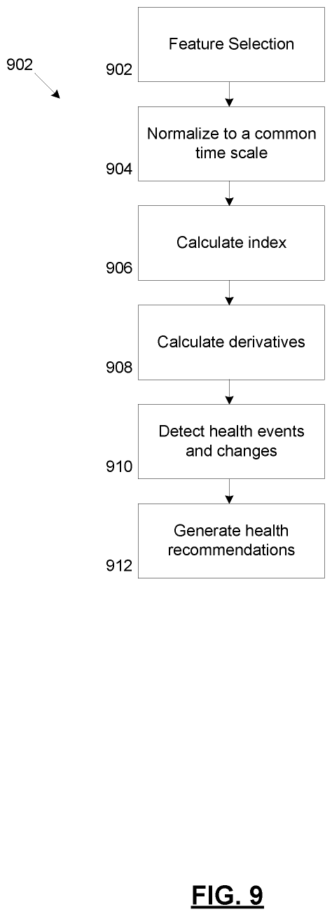

FIG. 9 illustrates an example method 900 for constructing and analyzing an allostatic load index AL(t), or another physiological index, from multi-modal data comprising physiological parameters, performing higher-order derivative analysis on the allostatic load index, detect derivative events, and converting those events into health recommendations, which may include real-time recommendations. The pipeline may be executed by the health recommendation unit on server 102 (see FIG. 2) and integrates with data model(s) 108 and user devices 104 (see FIG. 1).

At 902 the system selects a plurality of physiological parameters. The selected parameters may correspond to a parameters used to assemble an allostatic load index or another index. For example, stress related parameters available from wearables and clinical labs such as heart rate variability (HRV), resting heart rate (RHR), blood pressure (BP), cortisol (or surrogate), and sleep quality/duration may be selected. Other physiological parameters may also be included, e.g., temperature, activity regularity. The precise set may be selected or personalized based device capabilities and clinical goals. Precise timestamps are preserved to support irregular-sampling analysis in later stages.

At 904, the date for each physiological parameter is normalized to a common time scale and optionally direction-aligned (e.g., higher HRV→lower stress). Values for some parameters may be interpolated or otherwise estimated to provide value at particular timepoints so that an allostatic load (or other) index may be calculated at 906 at those timepoints. For example, the particular timepoints may be daily, weekly, monthly or on some other schedule. In some cases, some or all of the values for a physiological parameter may have been obtained at one or more of the timepoints, in which case the value may not be normalized and the raw value may be used for that parameter at that timepoint.

At 906, the time normalized physiological parameters are aggregated. The parameters may be aggregated at the timepoints for which data was normalized at 904 (including interpolated or otherwise estimated data for some of the parameters) to generate an index value according the allostatic load or other index. Physiological parameters may be weighted by relevance when calculating the index. For example, a particular approach to calculating an allostatic load index may assign a particular weight to each of the physiological parameters used to determine the index. In some embodiments, weights may be applied to parameters based on a particular person's health conditions, characteristics or goals, potentially providing a personalized index that may be used to assess the person's health and provide my personalized recommendations to the person.

At 908, the system calculates derivatives of the index. For example, for an allostatic load index AL(t), the system may calculate the first three derivatives, and optionally higher derivatives of the index:

$$A {L}^{′} ( t ) = \frac{d A L}{d t} , A {L}^{″} ( t ) = \frac{{d}^{2} A L}{d {t}^{2}} , A {L}^{′″} ( t ) = \frac{{d}^{3} A L}{d {t}^{3}} , etc .$$

At 910, the system detects changes or events in the index that may indicate health conditions or changes in health conditions. For example, the system may identify derivative events such as the index or one or more of the derivative values exceeding magnitude thresholds, as described above in relation to FIG. 7D, sign changes (for example, jerk turning from positive to negative indicating a deceleration in the rate of change of the index), and persistence windows (conditions sustained for N consecutive timepoints or T days).

At 912, the system generates health recommendations based on the changes or events detected at 912. Examples of health recommendations may include
Continue with a current intervention when jerk is relatively low indicating sustained stabilization (e.g., maintain sleep hygiene routine).
Adjust/Intensify interventions when jerk indicates erratic or concerning dynamics (e.g., increased HRV volatility+elevated BP).
Modify monitoring cadence (e.g., increase frequency during concerning phases; decrease frequency during stable phases).

The generated health recommendations may be displayed, sent or otherwise provided to a health care professional, clinician, a patient or another person with an interest in the person's health.

Combining multiple physiological parameters into an index, such as an allostatic load index and then applying higher order derivative analysis to the index may reduce the incidences of false health alerts while still being responsive to changes in the index and the underlying parameters to detect changes or events in a person's health.

Method 900 has been described as first calculating an index using time normalized values for physiological parameters, and then determining derivatives of the index value. In other embodiments, first, second, third and higher derivative values for individual parameters may first be estimated at particular timepoints as described above in relation to FIG. 6. The estimated derivative values for each parameter may then be combined to determine an index value at each timepoint.

Allostatic load (AL) quantifies the cumulative physiological ‘wear and tear’ that accrues as the body adapts to various stressors over time. Composite AL indexes may predict morbidity and mortality better than their individual components. The higher order derivative analysis describe above may also be used to analyse indices such as an allostatic load index.

An allostatic load index is typically built from a set of physiological parameters or parameters spanning cardiovascular, metabolic, and inflammatory systems (e.g., systolic/diastolic blood pressure, resting heart rate, total cholesterol, HDL, HbA1c, C-reactive protein (CRP), serum albumin), plus a central adiposity measure. Some allostatic load indices determine allostatic load either as a count-based score (number of parameters in “high-risk” ranges) or as a continuous score (summing normalized percentiles).

In some cases, the use of alternative physiological parameters may allow for improved detection of changes in health or the existence of health conditions. For example, some allostatic load indices use waist-to-hip ratio (WHR) as an estimate of a person's fat distribution and as a proxy for the amount of visceral fat that a person may have. However, such assumptions may be highly inaccurate for some people and will be somewhat inaccurate for many people. An alternative approach is to directly measure a person's body composition, including the amount and distribution of the person's visceral fat, and use the measured amount of visceral fat directly in calculating an allostatic index. Analyzing the resulting allostatic load index may result in earlier and more accurate identification of health changes and conditions.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention.

We claim:

1. A computer-implemented method for generating a health recommendation, comprising:
a. receiving, by one or more processors, time-stamped measurements of at least one physiological variable associated with an individual;
b. computing, from the measurements, at least a third derivative (jerk) of the physiological variable with respect to time;
c. detecting a derivative event based at least in part on the jerk, the derivative event comprising one or more of: (i) a magnitude-threshold condition, (ii) a sign change of the jerk, and (iii) satisfaction of a persistence window over a defined duration; and
d. in response to detecting the derivative event, generating a health recommendation selected from an intervention recommendation or a confirmation recommendation, and causing presentation of the health recommendation on a user device.

2. The method of claim 1, further including computing, from the measurements, at least one additional derivative and detecting the derivative event based, at least in part, on both the jerk and the at least one additional derivative.

3. The method of claim 1, wherein the physiological variable comprises one or more of LDL cholesterol, blood pressure, heart-rate variability, blood glucose, resting heart rate, sleep quality, skin temperature, or a composite index thereof.

4. The method of claim 1, wherein computing the derivatives comprises applying at least one of: secant-based first-derivative estimation, successive differences for higher derivatives, or analytic derivatives of a smooth fit to the measurements.

5. The method of claim 1, wherein detecting the derivative event comprises determining that an absolute jerk magnitude fall within a magnitude-threshold condition.

6. The method of claim 1, wherein the persistence window requires the jerk condition to be satisfied for a selected number of consecutive estimates or for at least a duration of time.

7. The method of claim 1, further comprising time-aligning data from heterogeneous sources and optionally applying smoothing using a moving average or spline prior to computing the derivatives.

8. The method of claim 1, wherein the health recommendation comprises continuing with current therapy when the jerk satisfies a decreasing or near-zero condition for the persistence window.

9. The method of claim 1, further comprising visualizing the physiological variable and at least one derivative with threshold overlays and pairing the visualization with the recommendation.

10. The method of claim 1, further comprising updating a monitoring cadence based on the derivative event.

11. The method of claim 1, wherein computing derivatives includes interpolating a derivative series to a regular evaluation grid to support daily recommendations.

12. The method of claim 1, wherein the measurements are irregularly sampled clinical values and the derivatives are computed using actual elapsed time between samples.

13. The method of claim 1, wherein the derivative event is detected on a composite index computed from multiple physiological variables.

14. The method of claim 1, wherein the recommendation includes intervention adjustment when the jerk indicates erratic or concerning dynamics.

15. The method of claim 1, wherein the method is performed by a server comprising a health recommendation unit configured to communicate with user devices via a network.

16. A computer-implemented method for estimating derivatives from irregularly sampled physiological data, comprising:
a. receiving time-stamped measurements (tᵢ, xᵢ) of a physiological variable with non-uniform intervals;
b. computing first-derivative estimates as secant slopes on intervals [tᵢ₋₁, tᵢ] and associating each first-derivative value with an interval midpoint;
c. computing second- and third-derivative estimates by successive differences between derivative values divided by actual elapsed time between corresponding derivative timestamps; and
d. interpolating at least one derivative series to arbitrary query times to support generation of time-normalized recommendations.

17. The method of claim 16, further comprising smoothing prior to differentiation using at least one of a moving average or a spline fit.

18. The method of claim 16, wherein interpolating comprises linear interpolation, a monotone interpolant, or a Hermite interpolant that preserves qualitative trends.

19. The method of claim 16, further comprising computing derivatives analytically from a smooth function S(t) fitted to (tᵢ, xᵢ), including S′(t), S″(t), and S″(t).

20. The method of claim 16, wherein the derivatives are provided to a derivative-event detector configured to apply threshold, sign-change, and persistence logic.
